(19) United States
(12) Patent Application Publication
(10) Pub. No.: US 2026/0273011 A1
(43) Pub. Date: Sep. 17, 2026

(54) COMPOSITION COMPRISING STEWARTIA PSEUDOCAMELLIA EXTRACT AS ACTIVE INGREDIENT FOR PREVENTION OR TREATMENT OF DEMENTIA

(71) Applicant: PUREBIO CO., LTD., Gyeongsangnam-Do (KR)

(72) Inventor: Woo Hyun NAM, Seoul (KR)

(21) Appl. No.: 18/877,787

(22) PCT Filed: Jun. 29, 2023
(86) PCT No.: PCT/KR2023/009094

§ 371 Date: Dec. 20, 2024

(30) Foreign Application Priority Data
Jul. 1, 2022 (KR) 10-2022-0081125

(51) Int. Cl. A61K 36/82; A61K 31/191; A61P 25/00; A61P 25/28; A61P 39/06

(52) U.S. Cl. CPC A61K 36/82; A61K 31/191; A61P 25/00; A61P 25/28; A61P 39/06; A61K 2236/19; A61K 2236/331; A61K 2236/333

(57) ABSTRACT

The present application provides a composition comprising a Stewartia pseudocamellia extract as an active ingredient for prevention or treatment of dementia.

### TECHNICAL FIELD

The present application relates to a composition including a Stewartia pseudocamellia extract and a use thereof. The present application claims the benefit of the date of application of Korean Patent Application No. 10-2022-0081125 filed to the Korean Intellectual Property Office on Jul. 1, 2022, the entire contents of which are incorporated herein by reference.

### BACKGROUND ART

Dementia is a common disease that occurs in approximately 8.2% to 10.8% of the elderly population aged 65 or older in Korea, and it is a representative geriatric disease that has emerged as a serious social problem along with the rapid aging of the population. Dementia is a degenerative disease that causes loss of intellectual ability and clinical symptoms such as cognitive impairment and gradual deterioration of behavior and personality. In particular, Alzheimer's disease (AD) is the most common type of dementia and is characterized by histological findings such as brain atrophy, senile plaques, and neurofibrillary tangles in the cerebral cortex or hippocampus, granulovacuolar degeneration of neurons, and Hirano bodies. Beta-amyloid (β-amyloid, Aβ) is a major component of senile plaques and is thought to be an important cause of AD. It is also known that Aβ induces neuronal cell death due to oxidative stress by generating reactive oxygen species (ROS).

The main symptom is cognitive function and memory decline due to brain cell destruction, and when it progresses slowly, it may lead to death. When it occurs between the ages of 40 and 65, it is classified as early-onset or familial AD, and when it occurs over the age of 65, it is classified as late-onset or sporadic AD. Familial AD is largely influenced by genetic factors. When dementia develops due to neuronal cell death caused by aging and progresses slowly, it is referred to as senile dementia, and AD is progressive, and the symptoms progress faster than senile dementia.

Meanwhile, in relation to such etiology, drugs developed so far to treat or improve dementia include antioxidants such as vitamin E and selegiline that suppress the destruction of brain cells by ROS, and acetylcholinesterase inhibitors such as tacrine, Aricept, and Exelon.

However, these drugs have disadvantages that they may cause side effects and their efficacy is not good. In addition, although many studies on dementia are currently being conducted in Korea and other countries, there has been no case of developing a new therapeutic that exhibits clear efficacy yet, and despite the fact that the influence of food intake is the greatest factor in the prevention of this disease, there is a lack of research on the food science approach.

### DISCLOSURE

### Technical Problem

A problem that the present application is to solve is to provide a composition for preventing or treating dementia, including a Stewartia pseudocamellia extract as an active ingredient.

Another problem that the present application is to solve is to provide a pharmaceutical composition for preventing or treating dementia, including a Stewartia pseudocamellia extract as an active ingredient.

Still another problem that the present application is to solve is to provide a food composition for preventing or ameliorating dementia, including a Stewartia pseudocamellia extract as an active ingredient.

Yet another problem that the present application is to solve is to provide a feed composition for preventing or ameliorating dementia, including a Stewartia pseudocamellia extract as an active ingredient.

Yet another problem that the present application is to solve is to provide a method for preventing or treating dementia by administering a pharmaceutical composition including a Stewartia pseudocamellia extract as an active ingredient to an individual other than a human.

Yet another problem that the present application is to solve is to provide a method for preventing or treating dementia by administering a composition including a Stewartia pseudocamellia extract as an active ingredient to a human.

Yet another problem that the present application is to solve is to provide a composition including a Stewartia pseudocamellia extract as an active ingredient for the purpose of preventing or treating dementia.

### Technical Solution

To solve the above-described problems, one embodiment of the present application provides a composition for preventing or treating dementia, including a Stewartia pseudocamellia extract as an active ingredient.

One embodiment of the present application provides a pharmaceutical composition for preventing or treating dementia, including a Stewartia pseudocamellia extract as an active ingredient.

One embodiment of the present application provides a food composition for preventing or ameliorating dementia, including a Stewartia pseudocamellia extract as an active ingredient.

One embodiment of the present application provides a feed composition for preventing or ameliorating dementia, including a Stewartia pseudocamellia extract as an active ingredient.

In one embodiment of the present application, the extract may be obtained using water, an organic solvent, or ultrasound.

In one embodiment of the present application, the extract may be obtained by fermenting a water extract with lactic acid bacteria (Lactobacillus plantarum).

In one embodiment of the present application, the extract may be an extract of the leaves or stems of Stewartia pseudocamellia.

In one embodiment of the present application, the extract may include ursolic acid.

In one embodiment of the present application, the dementia may be Alzheimer's dementia, cerebrovascular dementia, cerebral neuroinflammatory dementia, dementia with Lewy bodies (DLB), multi-infarct dementia (MID), frontotemporal lobar degeneration (FTLD), Pick's disease, corticobasal degeneration (CBD), progressive supranuclear palsy (PSP), cerebral amyloid angiopathy, Parkinson's disease, Huntington's disease, or mild cognitive impairment.

In one embodiment of the present application, the composition may suppress the activity of acetylcholinesterase and β-secretase.

In one embodiment of the present application, the composition may have antioxidant activity and suppress or eliminate the generation of reactive oxygen species.

In one embodiment of the present application, the composition may protect neurons against H₂O₂ cytotoxicity.

In one embodiment of the present application, the composition may suppress neuronal damage or cell death induced by the amyloid β-peptide.

### Advantageous Effects

A composition according to one embodiment of the present application has the effect of suppressing the activity of acetylcholinesterase, suppressing the activity of β-secretase, and having antioxidant activity. In addition, the composition has the effect of suppressing or eliminating the generation of reactive oxygen species and suppressing neuronal damage or cell death induced by the amyloid β-peptide, and thus can be utilized as a pharmaceutical composition and a food composition.

### DESCRIPTION OF DRAWINGS

FIG. 1 shows the 2,2′-azino-bis(3-ethylbenzothiazoline-6-sulfonic acid) (ABTS) radical scavenging activity and 2,2-diphenyl-1-picrylhydrazyl (DPPH) radical scavenging activity of the extract.

FIG. 2 shows the results of measuring the neurotoxicity of extract.

FIG. 3 shows the reactive oxygen species (ROS) generation suppressing activity of the extract.

FIG. 4 shows the acetylcholinesterase inhibiting activity of the extract.

FIG. 5 shows the β-secretase inhibiting activity of the extract.

FIG. 6 shows the neuronal cell death concentration for H₂O₂ cytotoxicity of the extract.

FIG. 7 shows the neuroprotective effect of the extract against H₂O₂ cytotoxicity.

FIG. 8 shows the neuronal cell death concentration for amyloid β-peptide 25-35 cytotoxicity of the extract.

FIG. 9 shows the neuroprotective effect of the extract against Aβ cytotoxicity.

FIG. 10 shows the ursolic acid content of samples.

FIG. 11 shows the ROS generation suppressing activity of ursolic acid.

FIG. 12 shows the neuroprotective effect of ursolic acid against H₂O₂ cytotoxicity.

FIG. 13 shows the neuroprotective effect of ursolic acid against Aβ cytotoxicity.

FIG. 14 shows the results of measuring the neurotoxicity of ursolic acid.

### MODES OF THE INVENTION

Hereinafter, the present application will be described in more detail.

The structural or functional descriptions of embodiments below are merely illustrated for the purpose of explaining embodiments according to the technical idea of the present application, and embodiments according to the technical idea of the present application may be implemented in various forms, and the technical idea of the present application should not be construed as being limited to the embodiments described herein.

Since the embodiments according to the technical idea of the present application can have various changes and can take various forms, specific embodiments are described in detail herein. However, this is not intended to limit the embodiments according to the technical idea of the present application to a specific disclosed form, and should be understood to include all modifications, equivalents, or substitutes included in the spirit and technical scope of the present application.

The terminology used herein is for the purpose of describing particular embodiments only and is not intended to limit the present application. Singular forms include plural forms, unless the context clearly indicates otherwise.

Unless otherwise defined, all terms, including technical and scientific terms used herein, have the same meaning as generally understood by one of ordinary skill in the art to which the present application pertains. Terms, such as those defined in commonly used dictionaries, should be interpreted as having a meaning that is consistent with their meaning in the context of the relevant art and are not interpreted in an idealized or overly formal sense unless clearly defined in the present application.

Stewartia pseudocamellia Maxim. is a deciduous tree of the family Theaceae, distributed in Jeonnam and Jeonbuk and Gyeongnam and Gyeongbuk in South Korea. It mainly grows in the Mt. Jiri area and is relatively abundant. This plant is 7 to 15 m tall, and its bark is reddish brown with a blackish tint, but the bark peels off in various places in a round or long pattern, exhibiting reddish yellow patterns. The leaves exhibit alternate phyllotaxy, have an oval shape, and the leaf margins have small serrations. The flowers are white and bloom where the leaves grow in June and July. The flowers are polypetalous, with five petals and many stamens. The hard fruit, which ripens in October, is covered with soft hair on the outside. Since the bark has a spotted pattern, the plant is also referred to as ‘silk tree.’ The leaves turn yellow in the fall, and the flowers look like camellia flowers, so it is often planted as an ornamental tree in gardens and parks. The wood is hard and used for furniture, and many people currently seek it out as a valuable medicine and tea. The bark is peeled off in thin pieces, forming reddish brown, grayish white, and grayish brown spotted patterns. The axils grow at the base of new branches, and the fruit is a pentagonal capsule. This plant is also referred to as Stewartia koreana Nakai or Stewartia pseudocamellia Maxim. var. koreana (Nakai) Kim, and some consider that it is an endemic species because its flowers are cup-shaped and its branches are straight and round. However, it is mainly noted as Stewartia pseudocamellia Maxim., which is currently distributed in Japan, and another synonym that has been documented is Stewartia ptero-petiolata Cheng var. koreana (Rehd.) Sealy. The flowers of Stewartia koreana are large and showy, so they are used for ornamental purposes. The wood is hard and has a beautiful pattern, so it is used for furniture and decorative materials. The bark and root bark are called ‘Moran’ and have the effect of relaxing muscles and promoting blood circulation, so they relieve blood stasis caused by bruises. Its activity related to rheumatism has also been studied.

The above-described Stewartia koreana extract may be extracted from various organs of the natural, hybrid, or mutant plant of Stewartia koreana, for example, the seeds, roots, aerial parts, stems, leaves, flowers, flesh of the fruit, peel of the fruit, as well as plant tissue cultures. Although not limited thereto, the Stewartia koreana extract may be at least one of a Stewartia koreana stem extract and a Stewartia koreana leaf extract.

The term “extract” herein means a product such as a liquid component obtained by immersing a target substance in one of various solvents and then extracting it for a certain time at room temperature, low temperatures, or heated conditions, or a solid component obtained by removing the solvent from the liquid component. In addition, it may be comprehensively interpreted to include a dilution of the product, a concentrate thereof, a crude substance, a purified substance, or the like in addition to the product.

The Stewartia koreana extract may be prepared using general extraction, separation, and purification methods known in the art. The extraction method may include, but is not limited to, immersion extraction, hot water extraction, cold immersion extraction, reflux cooling extraction, or ultrasonic extraction.

At this time, the organic solvent used is not particularly limited as long as it may be used to obtain an extract having an effect of preventing or treating cognitive dysfunction and neuroinflammation, but specifically, it may be water, a polar solvent, or a nonpolar solvent, and more specifically, it may be a solvent selected from the group consisting of water, lower alcohols having 1 to 6 carbon atoms (such as methanol, ethanol, propanol, or butanol), hexane, ethyl acetate, acetone, chloroform, and mixed solvents thereof, and most specifically, it may be ethanol or a mixed solvent thereof. In addition, a method for obtaining the extract is not particularly limited as long as it may be used to obtain an extract having an effect of preventing or treating dementia, but specifically, a cold extraction method in which the roots, stems, leaves, fruits, flowers, dried or processed products thereof of Stewartia koreana are immersed in the solvent and extracted at room temperature, a heat extraction method in which extraction is performed by heating to 40° C. to 100° C., an ultrasonic extraction method in which extraction is performed by applying ultrasonic waves, a reflux extraction method using a reflux condenser, or the like may be used.

The term “fraction” herein means a product obtained by a fractionation method for separating a specific component or a specific group from a mixture containing various components.

In the present application, the fraction may be interpreted as a fraction obtained by subjecting the Stewartia koreana extract to various fractionation methods. The fraction of the extract may be obtained by subjecting the extract to various fractionation methods, and the fractionation method is not particularly limited, but it may be a solvent fractionation method performed by treating the extract with various solvents, an ultrafiltration fractionation method performed by passing the extract through an ultrafiltration membrane having a constant molecular weight cut-off value, a chromatography fractionation method performed by performing various types of chromatography (designed for separation according to size, charge, hydrophobicity, or affinity), or the like.

In particular, the solvent used in the solvent fractionation method is not particularly limited, but a polar solvent or a nonpolar solvent may be used, and specifically, a nonpolar solvent may be used. The solvent fractionation method may be performed by sequentially fractionating the extract using a solvent having a high to low nonpolarity level, and for example, a method of sequentially fractionating the extract using nucleic acid or ethyl acetate may be used.

In addition, in the present invention, the fraction may be obtained by fractionation with a solvent selected from the group consisting of water, alcohol, hexane, ethyl acetate, acetone, chloroform, and mixed solvents thereof.

In general, dementia, as described in the above background art, refers to a disease in which impairment of cognitive function and personality changes occur later in life after maintaining a normal intellectual level during the growth period. Dementia is a disease in which the brain nerves are destroyed by various causes, resulting in overall mental function disorders such as memory impairment, language impairment, urinary incontinence, paranoid thinking, and aphasia. As dementia progresses, psychiatric symptoms such as depression, personality disorders, and aggression may also accompany it. The medical community is focusing on the causal factors of aging and genetics, but the exact cause and treatment method have not yet been identified.

Diseases that are classified as dementia may include, but are not limited to, Alzheimer's dementia, cerebrovascular dementia, cerebral neuroinflammatory dementia, dementia with Lewy bodies (DLB), multi-infarct dementia (MID), frontotemporal lobar degeneration (FTLD), Pick's disease, corticobasal degeneration (CBD), progressive supranuclear palsy (PSP), cerebral amyloid angiopathy, Parkinson's disease, Huntington's disease, or mild cognitive impairment, and the diseases that may be treated, prevented, or ameliorated by the composition provided by the present invention may include these diseases.

In this regard, the present technology can be used as a pharmaceutical material for treating dementia and a functional health food material because the Stewartia koreana extract has excellent anti-inflammatory activity, causes no cytotoxicity, inhibits the activity of acetylcholinesterase, inhibits the activity of β-secretase, has antioxidant activity, suppresses or eliminates the generation of reactive oxygen species (ROS), and has an excellent effect of suppressing neuronal damage or cell death induced by the amyloid β-peptide. In particular, “amyloid β-peptide (Aβ),” which is a protein produced from amyloid precursor protein by the action of an AD-specific protease, is known to cause the death of brain cells when deposited in brain tissue.

The present application provides a pharmaceutical composition for preventing or treating dementia, including a Stewartia pseudocamellia extract as an active ingredient.

The pharmaceutical composition may include a Stewartia pseudocamellia extract or a fraction thereof.

The term “prevention” as used herein refers to any action of suppressing or delaying the onset of dementia by administering the composition of the present invention.

The term “treatment” as used herein refers to an approach for acquiring beneficial or desirable clinical outcomes, including, but not limited to, alleviation of symptoms, reduction in the severity of a disease, stabilization (i.e., not worsening) of the disease, delay or slowing down the speed of disease progression, amelioration or palliation and alleviation of the disease conditions, whether the beneficial or desirable clinical outcomes are detectable or not, and whether they are partial or overall, for the purposes of the present invention.

Therefore, treatment refers to both therapeutic treatment and preventive treatment, and those that need to be treated include not only the state of already having a disease but also the state in which the disease needs to be prevented. In addition, treatment may refer to all actions that ameliorate the symptoms of dementia by administering the pharmaceutical composition of the present invention to an individual or benefit the individual.

In the present application, it is preferable to use 10% to 95% (v/v) of ethanol as a solvent for the preparation of the extract. More preferably, 50% to 95% (v/v) of ethanol may be used, and even more preferably, 70% to 95% (v/v) of ethanol may be used. In addition, it is preferable that the amount of the above-described solvent is 1 to 25 times the weight of the extraction raw material, and more preferably 5 to 20 times. The extraction temperature is preferably 50° C. to 90° C., and more preferably 60° C. to 80° C. The extraction time is preferably 1 to 12 hours, and more preferably 5 to 7 hours. According to these extraction conditions, the component exhibiting the effect targeted in the present invention may be efficiently extracted from the raw material.

The prepared extract may then be filtered, concentrated, or dried to remove the solvent, and filtration, concentration, and drying may all be performed. For example, filtration may be performed using filter paper or a reduced pressure filter, concentration may be performed using a vacuum concentrator, and drying may be performed using a freeze-drying method, but they are not limited thereto.

The ursolic acid according to the present application may be isolated from nature or prepared by a chemical synthesis method known in the art, preferably, it may be separated and purified from a Stewartia pseudocamellia extract, more preferably, from an ethanol extract of Stewartia pseudocamellia. The separation and purification of these compounds from the extract may be performed using column chromatography with columns filled with various synthetic resins, such as silica gel and activated alumina, and high-performance liquid chromatography (HPLC), alone or in combination. However, the extraction and separation and purification methods of the active ingredient are not necessarily limited to the above-described methods.

In addition, the composition according to the present application may include a pharmaceutically effective amount of the Stewartia pseudocamellia extract alone or may include one or more pharmaceutically acceptable carriers, excipients, or diluents. The pharmaceutically effective amount refers to an amount sufficient to prevent, ameliorate, and treat dementia symptoms.

The pharmaceutically effective amount of the Stewartia pseudocamellia extract according to the present application is 0.5 to 100 mg/day/kg of body weight, preferably 0.5 to 5 mg/day/kg of body weight. However, the pharmaceutically effective amount may be appropriately changed depending on the severity of dementia symptoms, the patient's age, weight, health conditions, and sex, administration route, and treatment period.

In addition, the term “pharmaceutically acceptable” as used herein refers to a composition that is physiologically acceptable and does not typically cause allergic reactions such as gastrointestinal disorders or dizziness or similar reactions when administered to humans. Examples of the carrier, excipient, and diluent include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil. In addition, fillers, anticoagulants, lubricants, wetting agents, fragrances, emulsifiers, and preservatives may be additionally included.

The pharmaceutical composition according to the present application may further include a pharmaceutically acceptable carrier in addition to the active ingredient. When the pharmaceutical composition is formulated, a pharmaceutical preparation may be prepared by mixing it with pharmaceutically acceptable excipients, adjuvants, analgesics, isotonic agents, preservatives, and other adjuvants and formulating it in a pharmaceutically acceptable dosage form, but is not limited thereto.

With regard the dosage form of such pharmaceutical preparations, the present application may be administered in various oral and parenteral dosage forms during actual clinical administration, and the most preferred administration route is oral administration. Solid preparations for oral administration may include tablets, pills, powder, granules, capsules, and the like. In addition, liquid preparations for oral administration include suspensions, solutions, emulsions, syrups, and the like, and preparations for parenteral administration (e.g., intravenous, subcutaneous, intraperitoneal, or topical application) include sterile aqueous solutions, non-aqueous solutions, suspensions, emulsions, lyophilized preparations, and suppositories, but may be appropriately selected by those skilled in the art.

The composition of the present application may be administered in combination with drugs that improve motor ability and prevent and restore cognitive function decline and improve daily life functions of patients with dementia, such as cholinesterase inhibitors such as tacrine, donepezil, rivastigmine, and galantamine; MAO-B inhibitors such as selegilin; vasodilators; and brain nutrients such as nootropics. In addition, when the composition of the present application is used in combination with haloperidol, which is an antipsychotic drug, clozapine, risperidone, and olanzapine known as atypical tranquilizers, and antidepressants such as Prozac, Zoloft, and Seroxat that inhibit serotonin reuptake, the amount of existing drugs used may be reduced and the problems of existing drugs may be alleviated, but the present application is not limited thereto.

In addition, the composition of the present application may be formulated using methods known in the art so as to provide rapid, sustained, or delayed release of the active ingredient after administration to a mammal. The formulation may be in the form of powder, granules, tablets, emulsions, syrups, aerosols, soft or hard gelatin capsules, sterile injectable solutions, or sterile powder.

The composition for preventing and treating dementia symptoms according to the present application may be administered via various routes including oral, transdermal, subcutaneous, intravenous, or intramuscular routes, and the dose of the active ingredient may be appropriately selected depending on various factors such as the administration route, the patient's age, sex, and weight, and the severity of a disease. In addition, the composition for preventing and treating dementia symptoms according to the present invention may be administered in combination with compounds having an effect of preventing, ameliorating, or treating dementia symptoms.

According to another aspect, the present application provides a food composition for preventing or ameliorating dementia, including a Stewartia pseudocamellia extract as an active ingredient.

The food composition for preventing or ameliorating dementia of the present application may include a Stewartia pseudocamellia extract or a fraction thereof.

The term “amelioration” as used herein refers to all actions that ameliorate the symptoms of an individual that is suspected of having or that has developed dementia, which is prevented or treated by using a composition including a Stewartia pseudocamellia extract or a fraction thereof as an effective ingredient, or benefit the individual.

Therefore, the composition for preventing or ameliorating dementia of the present application may be added to food for the purpose of preventing and ameliorating dementia symptoms, and thus the composition of the present invention may be used as a food composition for preventing and ameliorating dementia symptoms.

Therefore, the food composition for preventing or ameliorating dementia of the present application may be easily utilized as food that is effective in preventing and ameliorating dementia symptoms, such as a main ingredient or an auxiliary ingredient of food, a food additive, a functional food, or a beverage.

The food composition of the present application is very useful because it may be consumed on a daily basis and may be expected to have a preventive or ameliorating effect on cognitive dysfunction and neuroinflammation.

The term “food” as used herein refers to a natural product or processed product containing one or more nutrients, preferably means a product that has undergone a certain extent of processing to become immediately edible, and in its usual sense, includes all of foods, food additives, functional foods, and beverages.

Foods to which the composition for preventing or ameliorating dementia symptoms according to the present application may be added include, for example, various types of food, beverages, gum, tea, vitamin complexes, and functional foods. In addition, the foods in the present application include special nutritional foods (e.g., formulated milk, infant and toddler food, etc.), processed meat products, fish products, tofu, jelly, noodles (e.g., ramen, etc.), bread, health supplements, seasoned foods (e.g., soy sauce, soybean paste, red pepper paste, mixed sauce, etc.), sauces, confectioneries (e.g., snacks), candies, chocolates, gum, ice cream, processed dairy products (e.g., fermented milk, cheese, etc.), other processed foods, kimchi, pickled foods (various types of kimchi, pickled vegetables, etc.), beverages (e.g., fruit drinks, vegetable drinks, soy milk, fermented beverages, etc.), natural seasonings (e.g., ramen seasoning, etc.), but are not limited thereto. The foods, beverage, or food additive may be manufactured by a conventional manufacturing method.

In addition, the term “functional food” refers to a food group or food composition that has been designed and processed to sufficiently express the body's regulatory functions regarding biodefense rhythm regulation, disease prevention, and recovery, and the like, by using physical, biochemical, or bioengineering techniques to provide added value to the food so that the function of the food may be applied and expressed for a specific purpose, and specifically, it may be a health functional food. The functional food may include a foodologically acceptable food additive, and may further include appropriate carriers, excipients, and diluents commonly used in the manufacture of functional foods.

The term “health functional food” as used herein refers to foods manufactured (including processed) using raw materials or ingredients with useful functionality for the human body according to the Health Functional Foods Act, and the term “functionality” as used herein refers to obtaining a useful effect for health purposes such as regulating nutrients for the structure and function of the human body or physiological effects. Meanwhile, health food refers to a food that has a more active health maintenance or promotion effect than general foods, and health supplement food means a food for the purpose of health supplementation. In some cases, the terms, health functional food, health food, and health supplement food may be used interchangeably. The health functional food of the present application may be manufactured by a method commonly used in the art. It may be manufactured in various dosage forms, and unlike general drugs, it may have the advantage of causing no side effects that may occur when taking drugs for a long time because it uses food as a raw material, and it may be highly portable.

In addition, the term “beverage” as used herein is a general term for something that people drink to relieve thirst or enjoy a taste, and includes a functional beverage. The beverage has no special limitations on other ingredients except that it includes the composition for preventing and ameliorating dementia symptoms as an essential ingredient in the indicated proportion, and may contain various flavoring agents or natural carbohydrates as additional ingredients, like a common beverage.

Furthermore, in addition to those described above, a food containing the composition for preventing and ameliorating dementia symptoms of the present application may contain various nutrients, vitamins, minerals (electrolytes), flavoring agents such as synthetic flavoring agents and natural flavoring agents, coloring agents, and fillers (cheese, chocolate, etc.), pectic acid and salts thereof, alginic acid and salts thereof, organic acids, protective colloid thickeners, pH regulators, stabilizers, preservatives, glycerin, alcohol, carbonating agents used in carbonated beverages, and the like, and the above components may be used independently or in combination.

In the food containing the composition for preventing and ameliorating dementia symptoms of the present application, the amount of the composition according to the present invention may be included in an amount of 0.001% to 90% by weight, preferably 0.1% to 40% by weight of the total food weight, and in the case of a beverage, it may be included in an amount of 0.001 g to 2 g, preferably 0.01 g to 0.1 g, based on 100 ml. However, in the case of long-term intake for the purpose of health and hygiene or health control, the amount may be below the above-described range, and since the active ingredient does not have any problems in terms of safety, it may be used in an amount greater than the above-described range, and therefore is not limited to the above range.

One embodiment of the present application provides a feed composition for preventing or ameliorating dementia, including a Stewartia pseudocamellia extract or a fraction thereof as an active ingredient.

The feed composition may include a feed additive. The feed additive of the present application corresponds to supplementary feed under the Control of Livestock and Fish Feed Act.

The term “feed” as used herein refers to any natural or artificial diet, meal, etc., or a component of the meal, suitable for eating, ingesting, or digesting by an animal.

The type of feed is not particularly limited, and feed commonly used in the technical field may be used. Non-limiting examples of the feed include vegetable feed such as grains, roots, food processing by-products, algae, fibers, pharmaceutical by-products, fats, starches, meals, or grain by-products; and animal feed such as proteins, inorganic substances, fats, minerals, fats, single-cell proteins, zooplankton, or food. They may be used alone or in combination of two or more.

One embodiment of the present application provides a method for preventing or treating dementia by administering a pharmaceutical composition including a Stewartia pseudocamellia extract or a fraction thereof as an active ingredient to an individual other than a human.

One embodiment of the present application provides a method for preventing or treating dementia by administering a pharmaceutical composition including a Stewartia pseudocamellia extract as an active ingredient to a human.

The term “individual” as used herein refers to all animals, such as mice, livestock, and humans, that are likely to develop cognitive dysfunction or neuroinflammation or that have developed cognitive dysfunction or neuroinflammation.

Hereinafter, the present application will be described in more detail through examples, comparative examples, and experimental examples. The following examples are only intended to help understand the present application and do not limit the scope of the present application.

### <Preparation Example 1> Preparation of Extract

The leaves and stems of Stewartia pseudocamellia were dried in the shade, then each was ground and powdered. To prepare the extract, each type of powder was prepared, and 100% of the leaf+stem powder was used.

The hot water extract of Example 1 was prepared by mixing the powder with distilled water at a 1:20 ratio and extracting the mixture at 100° C. for six hours.

The ethanol extract of Example 2 was prepared by mixing the powder in with 95% ethanol at a 1:20 ratio and extracting the mixture under reflux at 65° C. for six hours.

The ultrasonic extract of Example 3 was prepared by mixing the powder with 95% ethanol at a 1:20 ratio and extracting the mixture at 40° C. for 30 minutes using an ultrasonic device (POWERSONIC 610, HWASHIN TECH, Daegu, Republic of Korea).

Lactic acid bacteria (Lactobacillus plantarum) were cultured in the hot water extract, 1% of the cell culture was inoculated, and fermented at 37° C. for 48 hours to prepare the lactic acid bacteria extract of Example 4.

The wolfberry was dried and powdered, and the resulting wolfberry powder was mixed with 95% ethanol as a solvent at a ratio of 1:20, and extracted under reflux for six hours at 65° C. to prepare the wolfberry extract of Comparative Example 1.

Among the extracts of Examples 1 to 4 and Comparative Example 1, the ethanol extract and the ultrasonic extract were filtered through a 3 μm filter and concentrated under reduced pressure, and the hot water extract and the lactic acid bacteria fermentation product were filtered through a 3 μm filter, and all extracts were freeze-dried and powdered.

### <Experimental Example 1> Antioxidant Activity

### 1. Measurement of 2,2′-Azino-Bis(3-Ethylbenzothiazoline-6-Sulfonic Acid) (ABTS) Radical Scavenging Activity of the Extracts

The ABTS radical scavenging activity was measured by modifying the method of Reet et al. (1999).

In other words, 5 mL of 7 mM ABTS and 88 μl of 140 mM potassium persulfate were mixed, and then the resulting mixture was blocked from light for 16 hours at room temperature to form ABTS cations. Thereafter, the solution was diluted with phosphate-buffered saline (PBS) so that the absorbance value at 414 nm was 1.5. After mixing the prepared diluted solution and a sample, the resulting mixture was allowed to react for six minutes at room temperature, and the absorbance was measured at 734 nm.

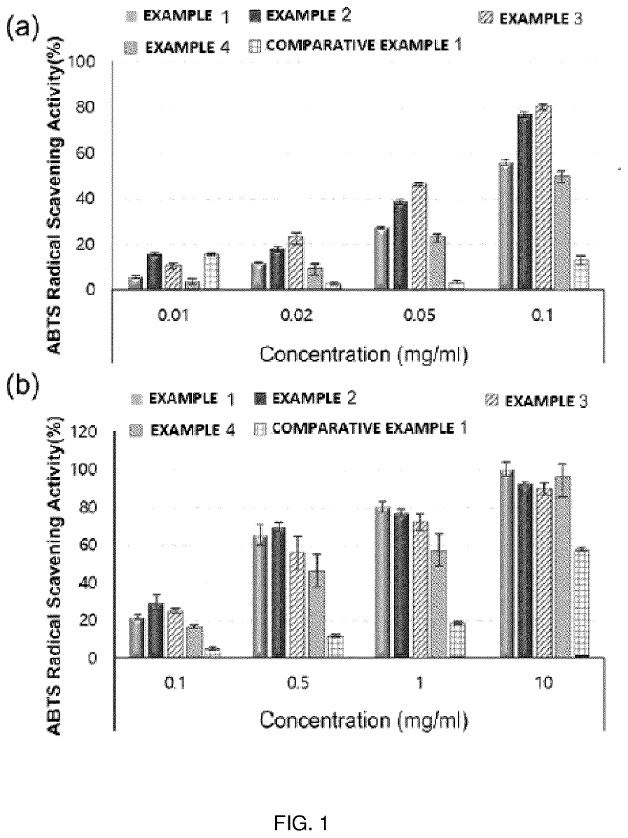

To determine the ABTS radical scavenging activity of the extracts prepared with each solvent, the percentage of the ABTS radical scavenging activity of the extract relative to that of the control group to which only the solvent was added was calculated according to Mathematical Equation 1, and the results are shown in FIG. 1A.

$$ABTS scavenging activity ( \% ) = \frac{\begin{matrix}( Absorbance of control - \\ Absorbance of sample )\end{matrix}}{Absorbance of control} \times 100 \qquad [ Mathematical Formula 1 ]$$

The ABTS radical scavenging activity of the ethanol extract of Example 2 and the ultrasonic extract of Example 3 was the highest at over 70%, and among them, the ultrasonic Stewartia pseudocamellia extract showed the highest scavenging activity at 80.0%. The wolfberry extract of Comparative Example 1 exhibited 12.6% ABTS radical scavenging activity.

### 2. Measurement of 2,2-Diphenyl-1-Picrylhydrazyl (DPPH) Radical Scavenging Activity of the Extracts

A DPPH assay was performed according to the method used by Yoshida et al. (1989).

A solvent was added to the control group and a sample was added to the test group in a 96-well plate. 50 μl of a DPPH (0.5 mM 1,1-diphenyl-2-picrylhydrazyl (DPPH)/ethanol) solution was added to all wells and mixed. After a reaction at room temperature of 25° C. for 30 minutes, the absorbance was measured at 517 nm.

To determine the DPPH radical scavenging activity of the extracts prepared with each solvent, the percentage of DPPH radical scavenging activity of the extract relative to that of the control group to which only the solvent was added was calculated according to Mathematical Equation 2, and the results are shown in FIG. 1B.

$$DPPH scavenging activity ( \% ) = \frac{\begin{matrix}( Absorbance of control - \\ Absorbance of sample )\end{matrix}}{Absorbance of control} \times 100 \qquad [ Mathematical Formula 2 ]$$

DPPH radical scavenging activity did not differ significantly depending on the extraction method. When the concentration was 10 (mg/ml), the hot water extract of Example 1 showed 100% DPPH radical scavenging activity, the ethanol extract of Example 2 showed 92.2%, the ultrasonic extract of Example 3 showed 90.1%, the lactic acid bacteria fermentation product of Example 4 showed 96.6%, and the wolfberry extract of Comparative Example 1 showed 57.1%.

### <Experimental Example 2> ROS Generation Suppression Ability of the Extracts in Neurons

### 1. Human Neuroblastoma SH-SY5Y Cell (VA 20108) Culture

Neuronal cells (human neuroblastoma SH-SY5Y) were obtained from the Korean Cell Line Bank (KCLB, Seoul, Korea). The medium used was a minimum essential medium (SH30024.01, Cytiva, USA) containing 10% fetal bovine serum (FBS, Gibco, CA, USA) medium, 25 mM 4-(2-hydroxyethyl)-1-piperazine ethanesulfonic acid (HEPES), and 25 mM NaHCO₃. The cells were cultured in an incubator controlled at 95% humidity, 5% CO₂, and 37° C., and an antibiotic for the medium (penicillin-streptomycin, Gibco, CA, USA) was used to suppress microbial contamination or proliferation. When the cells covered about 80% of the dish, the dish was washed with phosphate-buffered saline-ethylenediaminetetraacetic acid (PBS-EDTA, Thermo Fisher Scientific, Massachusetts, USA), and then 1 ml of 0.05% trypsin-EDTA (Gibco, Thermo Fisher Scientific, Massachusetts, USA) was used to detach the cells attached to the bottom of the dish and subculture them. The medium was exchanged with fresh medium every 48 to 72 hours to culture the cells.

### 2. Human Neuroblastoma SH-SY5Y Cell (VA 20108) Toxicity

The cells were plated into a 96-well plate at a concentration of 5×10³ cells/well, and then cultured for 24 hours at 95% humidity, 5% CO₂, and 37° C. for stabilization. After removing the medium, 100 μl of the sample diluted with fresh medium was applied and allowed to react with the cells for 24 hours in an incubator at 95% humidity, 5% CO₂, and 37° C.

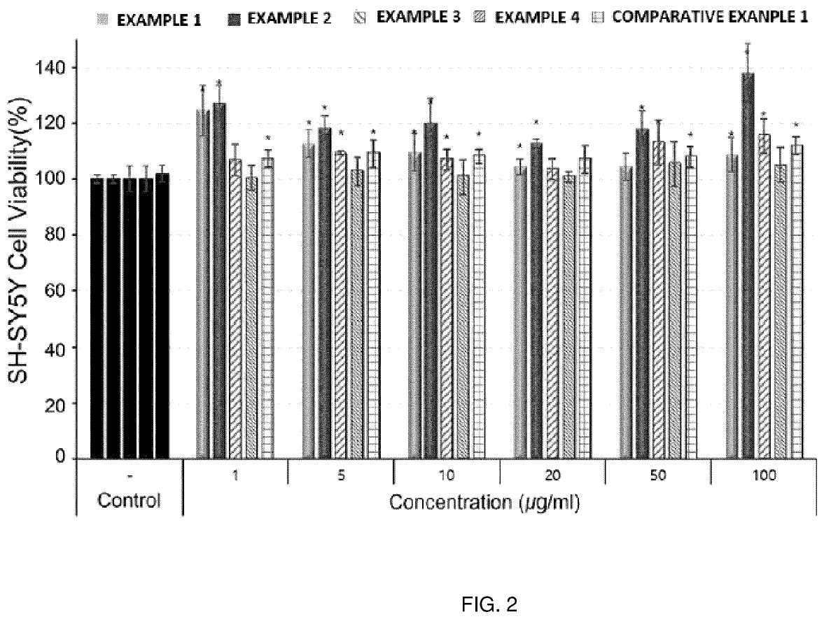

The viability of the cells was measured using the 3-(4,5-dimethylthiazol-2-yl)-5-(3-carboxymethoxyphenyl)-2-(4-sulfophenyl)-2H-tetrazolium (MTS, CellTiter 96® AQueous One Solution Cell Proliferation Assay, Promega Inc., USA) reagent. In other words, 1 ml of the reagent was diluted with 9 ml of medium (DMEM high glucose, free FBS), and 100 μl of the mixture was added to each well. After a reaction at 37° C. for 60 minutes, the absorbance was measured at 490 nm using a microplate reader (Molecular Devices LLC, VersaMax ELISA Microplate Reader, USA). The cell viability was expressed as the absorbance of the sample-treated group relative to that of the untreated control group, and the results are shown in FIG. 2.

The concentration for measuring the ROS generation suppression activity and the cell protection effect was 1 to 100 μg/ml, and the results of treating the cells showed that the Stewartia pseudocamellia extract and the wolfberry extract exhibited no toxicity to the neurons even at high concentrations.

### 3. ROS Generation Suppression Ability in Neurons

The generation of ROS is measured by the intensity of fluorescent material generation using 2′,7′-dichlorofluorescin diacetate (DCF-DA), and the principle is that 2′,7′-dichlorofluorescin (DCF) in a reduced or acetylated form releases a fluorescent material (fluorescein isothiocyanate, FITC) when the acetate group is removed by the oxidation of esterase and ROS generated within cells.

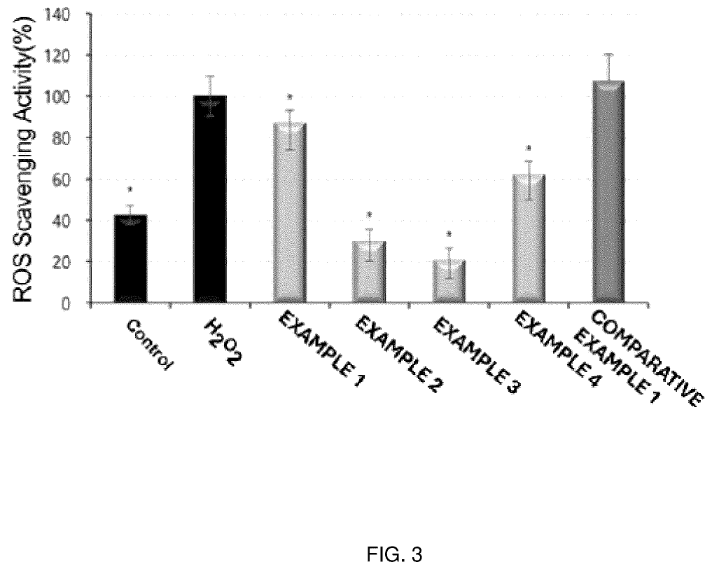

SH-SY5Y cells were plated into a 96-well plate (100 μl) at a concentration of 5×10⁴ cells/well and cultured for 24 hours. After the culture, the medium was removed, and the sample was treated at an appropriate concentration using a medium containing no serum and then cultured for 24 hours. After 24 hours, 100 μl of PBS was added and the resulting mixture was washed, and this process was repeated three times. DCF-DA dissolved in dimethyl sulfoxide (DMSO) was diluted to 20 μM (in PBS), and 100 μl of the diluted solution was added and incubated for 20 minutes. After adding 100 μl of 100 μM H₂O₂ (in PBS), the mixture was allowed to react for 30 minutes, and the fluorescence intensity was measured at 486 nm and 535 nm using an enzyme-linked immunosorbent assay (ELISA) reader. The ROS generation suppression ability of the extracts is shown in FIG. 3.

All samples were measured at a concentration of 1 to 100 μg/ml, and at a concentration of 100 μg/ml, the hot water extract of Example 1 showed 13% ROS generation suppression ability, the ethanol extract of Example 2 showed 70%, the ultrasonic extract of Example 3 showed 79%, and the lactic acid bacteria fermentation product of Example 4 showed 38%. The wolfberry extract of Comparative Example 1 showed 0%, indicating that it has no ROS generation suppression ability.

### <Experimental Example 3> Measurement of Acetylcholinesterase Inhibition Activity of the Extracts

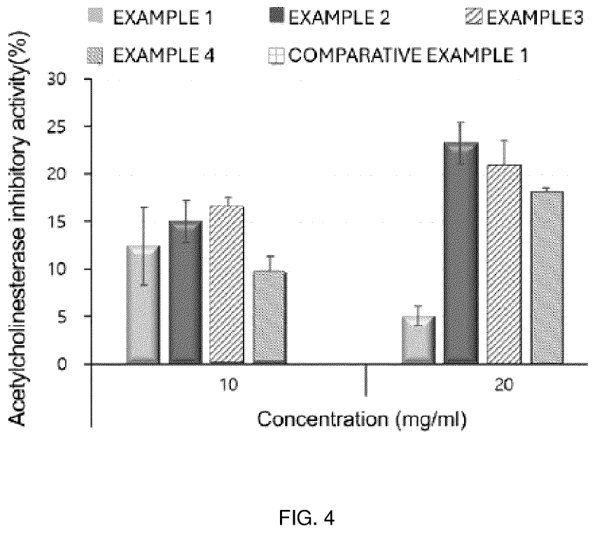

The acetylcholinesterase inhibition activity of the Stewartia pseudocamellia extract was measured using the acetylcholinesterase assay kit (colorimetric) manufactured by Abcam with a slightly modified method as described below. 50 μL of the mixture (assay buffer 4.5 mL+20×5,5′-dithiobis(2-nitrobenzoic acid (DTNB) stock solution 250 μL+20× acetylcholine stock solution 250 μL=5 mL), 50 μl of acetylcholinesterase (0.5 unit/mL), and 5 μl of the sample were added to a 96-well plate, and the resulting mixture was allowed to react for 30 minutes at room temperature, and then the absorbance was measured at 410 nm using a microplate reader (Molecular Devices LLC, VersaMax ELISA Microplate Reader, USA). The enzyme inhibition activity of the extract relative to that of the control group to which only the solvent was added was expressed as a percentage according to the following Mathematical Formula 3, and the results are shown in FIG. 4.

$$Acetylcholinesterase inhibition activity ( \% ) = ( 1 - \frac{\begin{matrix}( Absorbance of sample - \\ Absorbance of control )\end{matrix}}{\begin{matrix}Absorbance of “ enzyme - \\ substrate ” - Absorbance of control\end{matrix}} ) \times 100 \qquad [ Mathematical Formula 3 ]$$

All samples were measured at a concentration of 10 to 20 mg/ml, and at a concentration of 20 mg/ml, the hot water extract of Example 1 showed 5% acetylcholinesterase inhibition activity, the ethanol extract of Example 2 showed 23.2%, the ultrasonic extract of Example 3 showed 20.8%, and the lactic acid bacteria fermentation product of Example 4 showed 18.1%, while the wolfberry extract of Comparative Example 1 showed 0%, indicating that it has no acetylcholinesterase inhibition activity.

### <Experimental Example 4> β-Secretase Inhibition Activity of the Extracts

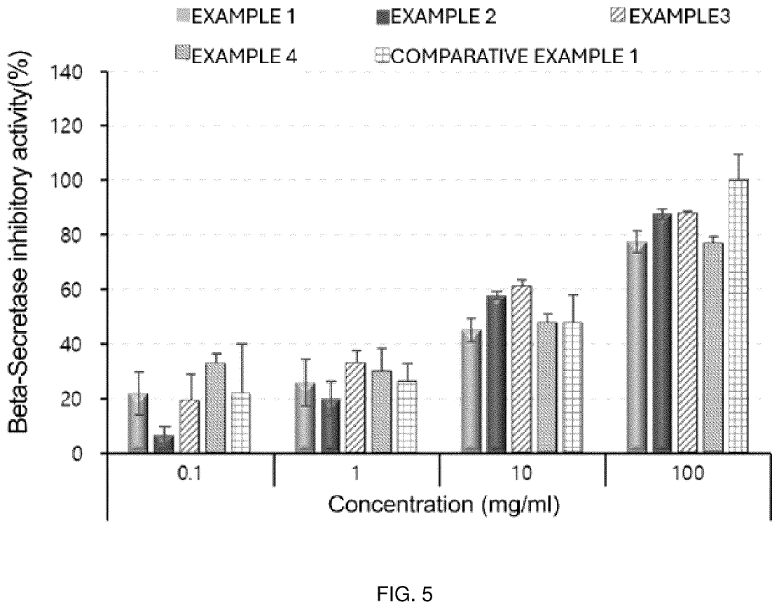

The β-secretase inhibition activity of the Stewartia pseudocamellia extract was measured as follows using the β-secretase (BACE1) activity assay kit (fluorometric) manufactured by Abcam with a slightly modified method. 5 μl of the Stewartia pseudocamellia extract, 2 μl of the BACE1 substrate, 83 μL of the assay buffer, and 10 μl of the 10 μM N-(2,4-dinitrophenyl)-N′-(9-anthryl oxycarbonyl)-ethylenediamine (EDANS) standard were added to a 96-well plate, and the resulting mixture was allowed to react at 25° C. for 30 minutes. The fluorescence intensity was measured at 355 nm (excitation) and 460 nm (emission) using a multilabel plate reader (PerkinElmer 2030 multilabel reader, PerkinElmer, USA). The enzyme inhibition activity of the extract relative to that of the control group to which only the solvent was added was expressed as a percentage according to the following Mathematical Formula 4, and the results are shown in FIG. 5.

$$β - Secretase inhibition activity ( \% ) = ( 1 - \frac{\begin{matrix}( Absorbance of sample - \\ Absorbance of control )\end{matrix}}{\begin{matrix}Absorbance of “ enzyme - \\ substrate ” - Absorbance of control\end{matrix}} ) \times 100 \qquad [ Mathematical Formula 4 ]$$

All samples were measured at concentrations of 0.1 to 100 mg/ml, and at a concentration of 100 mg/ml, the hot water extract of Example 1 showed 77.5% β-secretase inhibition activity, the ethanol extract of Example 2 showed 87.4%, the ultrasonic extract of Example 3 showed 87.9%, the lactic acid bacteria fermentation product of Example 4 showed 76.9%, and the wolfberry extract of Comparative Example 1 showed 100%.

### <Experimental Example 5> Measurement of Neuroprotective Effect of the Extracts Against H₂O₂ Cytotoxicity

1. Measurement of Cell Death Concentration for H₂O₂

Hydrogen peroxide 30% (DAEJUNG, 4104-4405) was purchased and used to measure the neuronal cell death concentration by H₂O₂.

First, the cells were plated into a 96-well plate at a concentration of 5×10³ cells/well, and then cultured for 24 hours at 95% humidity, 5% CO₂, and 37° C. for stabilization. After removing the medium, 10 μl of H₂O₂ diluted with fresh medium was treated at each concentration, and the resulting mixture was allowed to react for 24 hours at 95% humidity, 5% CO₂, and 37° C.

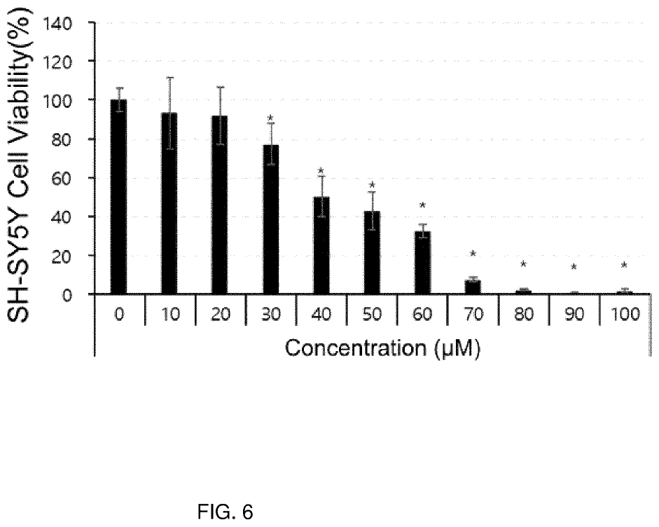

The viability of the cells was measured using the MTS (CellTiter 96® AQueous One Solution Cell Proliferation Assay, Promega, USA) reagent. In other words, 10 μl of the reagent was treated per well, and after a reaction at 37° C. for 60 minutes, the absorbance was measured at 490 nm using a microplate reader (Molecular Devices LLC, VersaMax ELISA Microplate Reader, USA). The cell viability was expressed as the absorbance of the sample-treated group relative to that of the untreated control group, and the results are shown in FIG. 6.

To measure the cell death concentration, H₂O₂ was treated at a concentration of 10 to 100 μM, and it was confirmed that the cell viability was approximately 50% at a concentration of 50 μM.

2. Neuroprotective Effect Against H₂O₂ Cytotoxicity

After plating the cells into a 96-well plate at a concentration of 5×10³ cells/well, the cells were cultured at 95% humidity, 5% CO₂, and 37° C. for 24 hours for stabilization. After removing the medium, 50 μM H₂O₂, which has a cell viability of about 50%, was treated with fresh medium, and then 10 μl of the samples of Examples 1 to 4 and Comparative Example 1 were treated at each concentration, and the resulting mixtures were allowed to react at 95% humidity, 5% CO₂, and 37° C. for 24 hours.

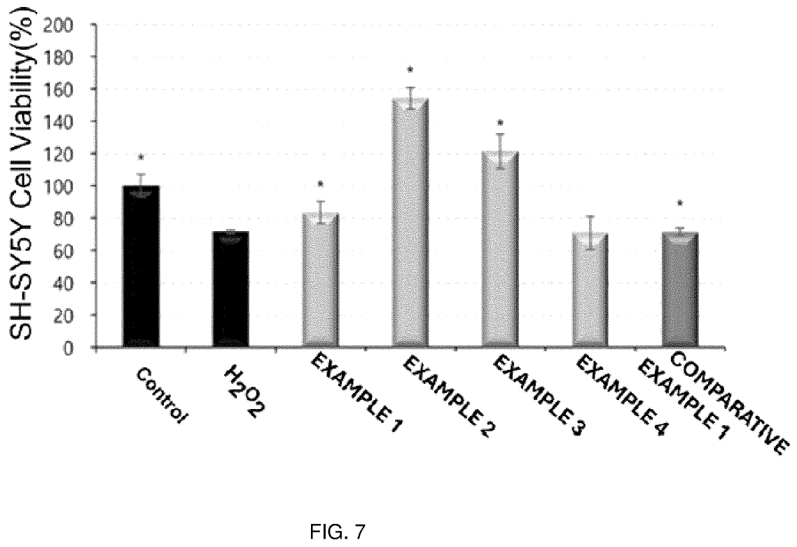

The cell viability was measured using the MTS (CellTiter 96® AQueous One Solution Cell Proliferation Assay, Promega, USA) reagent. In other words, 10 μl of the reagent was treated per well, and after reaction at 37° C. for 60 minutes, the absorbance was measured at 490 nm using a microplate reader (Molecular Devices LLC, VersaMax ELISA Microplate Reader, USA). The cell viability was expressed as the absorbance of the sample-treated group relative to that of the untreated control group, and the results are shown in FIG. 7.

The cell protective effect was observed at a concentration of 10 μg/ml or higher, and at a concentration of 100 μg/ml, the hot water extract of Example 1 showed a cell viability of 83.3%, the ethanol extract of Example 2 showed 154.1%, the ultrasonic extract of Example 3 showed 121.3%, the lactic acid bacteria fermentation product of Example 4 showed 70.9%, and the wolfberry extract of Comparative Example 1 showed 71.5%, confirming cell protection efficacy.

### <Experimental Example 6> Measurement of the Neuroprotective Effect of the Extracts Against Amyloid β (Aβ)-Peptide 25-35 Cytotoxicity

### 1. Measurement of Cell Death Concentration for Aβ-Peptide 25-35

To measure the neuronal cell death concentration by Aβ 25-35, Aβ-protein fragment 25-35 (Sigma-Aldrich, A4559-1 MG) was purchased and used.

For the aggregation of Aβ, it was dissolved in distilled water and allowed to react at 37° C. for five days at 150 rpm.

First, the cells were plated into a 96-well plate at a concentration of 5×10³ cells/well, and then cultured at 95% humidity, 5% CO₂, and 37° C. for 24 hours for stabilization. After removing the medium, 10 μl of Aβ diluted in fresh medium was treated at each concentration, and the resulting mixture was allowed to react at 95% humidity, 5% CO₂, and 37° C. for 24 hours.

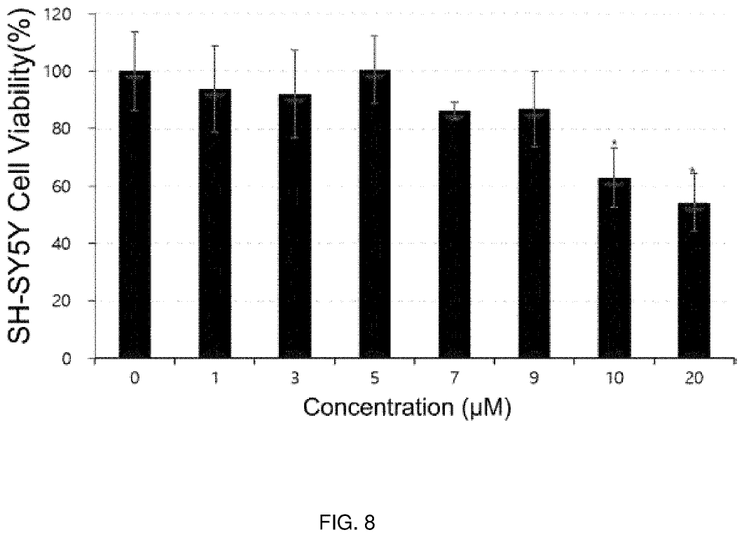

The viability of the cells was measured using the MTS (CellTiter 96® AQueous One Solution Cell Proliferation Assay, Promega, USA) reagent. In other words, 10 μl of the MTS reagent was treated per well, and after a reaction at 37° C. for 60 minutes, the absorbance was measured at 490 nm using a microplate reader (Molecular Devices, VersaMax ELISA Microplate Reader, USA). The viability of the cells was expressed as the absorbance of the sample-treated group relative to that of the untreated control group, and the results are shown in FIG. 8.

Aβ was treated at a concentration of 1 to 20 μM, and the cell viability was confirmed to be approximately 50% at a concentration of 20 μM.

### 2. Neuroprotective Effect Against Aβ-Peptide 25-35 Cytotoxicity

The cells were plated into a 96-well plate at a concentration of 5×10³ cells/well and cultured at 95% humidity, 5% CO₂, and 37° C. for 24 hours for stabilization. After removing the medium, 20 μM Aβ, which has a cell viability of about 50%, was treated with fresh medium, and 10 μl of the samples of Examples 1 to 4 and Comparative Example 1 were treated at each concentration, and the resulting mixture was allowed to react at 95% humidity, 5% CO₂, and 37° C. for 24 hours.

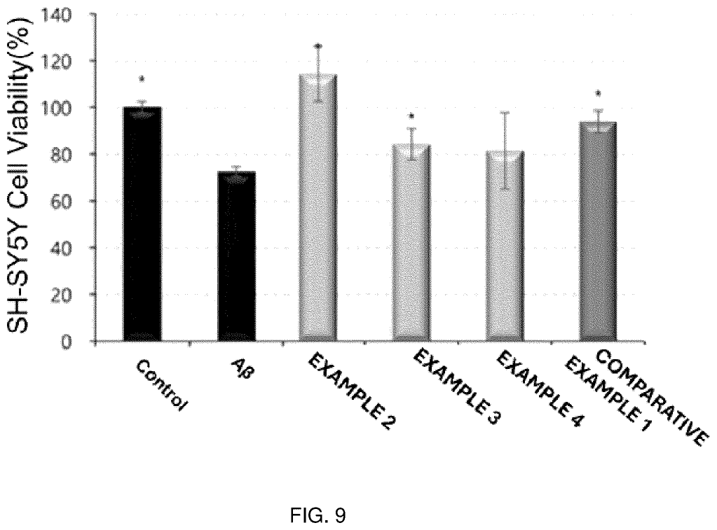

The cell viability was measured using MTS (CellTiter 96® AQueous One Solution Cell Proliferation Assay, Promega, USA) reagent. In other words, 10 μl of the MTS reagent was treated per well, and after a reaction at 37° C. for 60 minutes, the absorbance was measured at 490 nm using a microplate reader (Molecular Devices, VersaMax ELISA Microplate Reader, USA). The cell viability was expressed as the absorbance of the sample-treated group relative to that of the untreated control group, and the results are shown in FIG. 9.

It was confirmed that at a concentration of 100 μg/ml, the ethanol extract of Example 2 showed a cell viability of 114.2%, the ultrasonic extract of Example 3 showed 84.2%, the lactic acid bacteria fermentation product of Example 4 showed 81.3%, and the wolfberry extract of Comparative Example 1 showed 93.9%.

### <Experimental Example 7> Analysis of Ursolic Acid as Marker Substance Content in the Stewartia pseudocamellia Extract

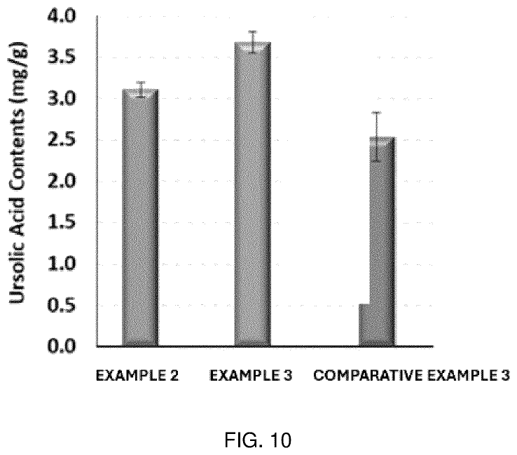

Among the Stewartia pseudocamellia extracts, the ethanol extract of Example 2 and the ultrasonic extract of Example 3 showed high ursolic acid contents of 0.31% and 0.37%, respectively. The wolfberry extract of Comparative Example 1 showed an ursolic acid content of 0.25%. The ursolic acid content of the samples is shown in FIG. 10.

### <Experimental Example 8> ROS Generation Suppression Ability of Ursolic Acid in Neurons

### 1. Human Neuroblastoma SH-SY5Y Cell (VA 20108) Toxicity

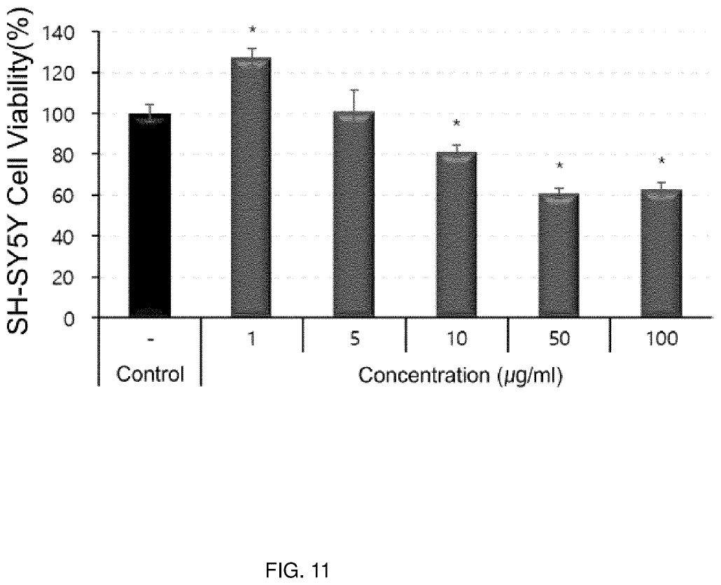

As a result of measuring the cytotoxicity of ursolic acid on neurons, toxicity was exhibited at a concentration of 10 μg/ml or higher, so the concentration for measuring the ROS generation suppression ability and the neuroprotective effect was set to 1 to 5 μg/ml. The results of measuring the neurotoxicity of ursolic acid are shown in FIG. 11.

### 2. ROS Generation Suppression Ability in Neurons

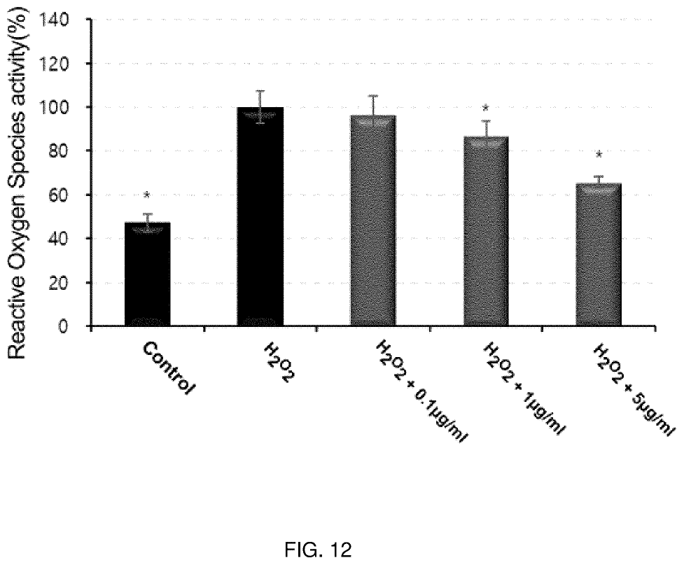

As a result of measuring the ROS generation suppression ability using neurons (SH-SY5Y), significant suppression activity was observed at ursolic acid concentrations of 0.1 to 5 μg/ml. The ROS generation suppression activity of ursolic acid is shown in FIG. 12.

### <Experimental Example 9> Measurement of the Neuroprotective Effect of Ursolic Acid Against H₂O₂ Cytotoxicity

1. Neuroprotective Effect Against H₂O₂ Cytotoxicity

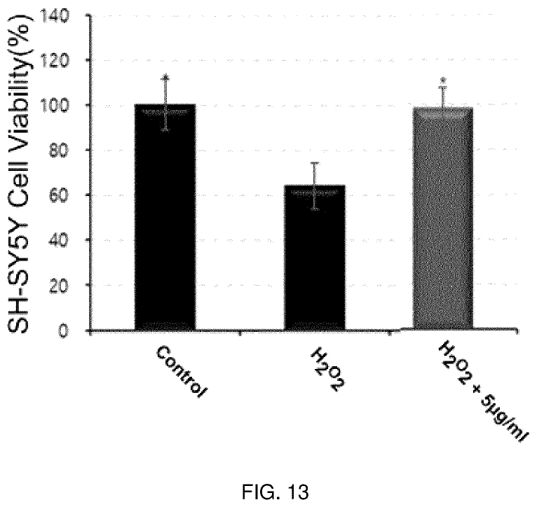

As a result of the experiment on the neuroprotective effect by 50 mM H₂O₂ treatment, when the cells were treated with ursolic acid, cell proliferation was similar to that of the H₂O₂ untreated group, confirming high cell protection efficacy. The neuroprotective effect of ursolic acid against H₂O₂ cytotoxicity is shown in FIG. 13.

### <Experimental Example 10> Measurement of the Neuroprotective Effect of Ursolic Acid Against Aβ-Peptide 25-35 Cytotoxicity

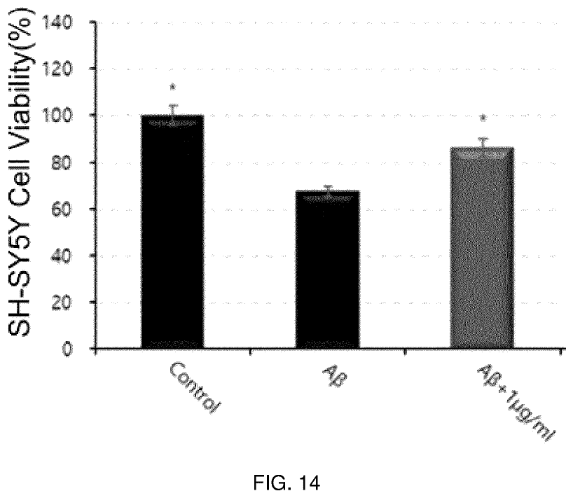

As a result of the experiment on the neuroprotective effect by 20 mM Aβ treatment, ursolic acid exhibited a significant effect. The neuroprotective effect of ursolic acid against Aβ cytotoxicity is shown in FIG. 14.

Although specific parts of the present application have been described in detail above, it is obvious to those skilled in the art that such specific descriptions are merely preferred examples and that the scope of the present application is not limited thereto. Therefore, the substantial scope of the present application is defined by the appended claims and their equivalents.

1. A composition for preventing or treating dementia, comprising a Stewartia pseudocamellia extract as an active ingredient.

2. The composition according to claim 1, wherein the extract is a water extract, an organic solvent extract, or an ultrasonic extract, or is obtained by fermenting a water extract with lactic acid bacteria of Lactobacillus plantarum.

3. The composition according to claim 1, wherein the extract includes ursolic acid.

4. The composition according to claim 1, wherein the dementia is Alzheimer's dementia, cerebrovascular dementia, cerebral neuroinflammatory dementia, dementia with Lewy bodies (DLB), multi-infarct dementia (MID), frontotemporal lobar degeneration (FTLD), Pick's disease, corticobasal degeneration (CBD), progressive supranuclear palsy (PSP), cerebral amyloid angiopathy, Parkinson's disease, Huntington's disease, or mild cognitive impairment.

5. The composition according to claim 1, wherein the composition suppresses the activity of acetylcholinesterase and β-secretase.

6. The composition according to claim 1, wherein the composition has antioxidant activity and suppresses or eliminates the generation of reactive oxygen species.

7. The composition according to claim 1, wherein the composition protects neurons against H₂O₂ cytotoxicity and suppresses neuronal damage or cell death induced by amyloid β-peptide.

8. A food or feed composition for preventing or ameliorating dementia, comprising a Stewartia pseudocamellia extract as an active ingredient.

9. (canceled)

10. A method for preventing or treating dementia, comprising:
administering to a subject in need thereof a Stewartia pseudocamellia extract.

11. (canceled)
